(19) United States
(12) Patent Application Publication
(10) Pub. No.: US 2026/0301863 A1
(43) Pub. Date: Oct. 1, 2026

(54) Systems and methods for performing Reverse-Correlated Multiphasic Analysis

(71) Applicant: Arun Christopher Konanur, London (CA)

(72) Inventor: Arun Christopher Konanur, London (CA)

(21) Appl. No.: 19/093,947

(22) Filed: Mar. 28, 2025

(51) Int. Cl. G16B 20/20; G16B 30/10

(52) U.S. Cl. CPC G16B 20/20; G16B 30/10

(57) ABSTRACT

A bioinformatic system that facilitates the identification of the common ancestral connection of minimally correlated autosomal DNA (atDNA) matches is disclosed. The invention consists of two main components: The first is Reverse-Correlated Multiphasic Analysis (Reverse-CMA or R-CMA) a process of comparing a designated In Common With (ICW) atDNA match (the R-CMA Nexus) against a subset of the atDNA matches (Test Subjects) used to initially identify the R-CMA Nexus, in order to derive a collection of individuals whose Most Recent Common Ancestral Couple (MRCAC) may suggest an ancestral connection between the Test Subjects and the R-CMA Nexus itself. The second is a set of automated scripts, formulae, and data structures to facilitate desktop correlation and tabulation utilizing R-CMA in conjunction with a desktop spreadsheet program such as Microsoft Excel. These data structures and formulae may be adapted to facilitate the deployment of R-CMA within an enterprise-level database management system.

### FIELD OF THE INVENTION

The present invention relates to a system that performs Reverse-Correlated Multiphasic Analysis (R-CMA), a method of organizing autosomal DNA matches, both on a personal (desktop spreadsheet tabulation) and on an enterprise (database management system) platform.

### BACKGROUND OF THE INVENTION

Correlated Multiphasic Analysis (CMA) (USPTO application # Ser. No. 17/470,321), is a bioinformatic system that identifies the common ancestral origins of otherwise uncorrelated autosomal DNA (atDNA) matches, and delivers powerful insights drawn from the totality of a subject's atDNA results.

CMA generates an Analytic Control Set (ACS) which delivers two types of actionable intelligence:
the ACS may be sorted by Genetic Complex—collections of individuals organized about a Most Recent Common Ancestral Couple (MRCAC)—which may be validated using the SMD process (USPTO application # Ser. No. 18/793,774) and further investigated using the AASK process (USPTO application # Ser. No. 18/641,045).
the ACS may also be sorted by criteria which rank individual DNA matches by granular properties, such as the amount of DNA shared with CMA's Test Subjects, or the number of Test Subjects each element of the ACS matches, corresponding to the magnitude of that individual element's ACS Classification. Reverse-CMA was developed to facilitate the investigation of individual ACS elements with an ACS Classification of large magnitude.

Reverse-CMA was developed to investigate the ancestral connection between individual elements of the ACS and the collection of Test Subjects from which the ACS itself is generated—the idea being that, while the AASK process requires hundreds (potentially even thousands) of sets of DNA matches in order to organize and stratify an entire Genetic Complex, only one additional set of DNA matches is required for Reverse-CMA to generate actionable intelligence, which may in turn be used to assign an ACS element and its In Common With matches to a newly derived Genetic Complex.

### SUMMARY OF THE INVENTION

This invention is directed to generate actionable intelligence—information which may be investigated further and genealogically verified through traditional research—regarding the ancestral connection between an element of CMA's Analytic Control Set (the R-CMA Nexus) and the Test Subjects whose In Common With DNA matches were used to generate the ACS.

Although Reverse-CMA may be employed to investigate any element of the Analytic Control Set, common sense suggests that the most fruitful use of Reverse-CMA lies in the investigation of those elements of the ACS with significant connections to the CMA test subjects themselves. This protocol is facilitated by the sorting functions in the CMA Master Workbook, which can sort the elements of the Analytic Control Set by the magnitude of their ACS Classification (|ACS Class|).

### BRIEF DESCRIPTION OF THE DRAWINGS

In order to facilitate a fuller understanding of the present invention, reference is now made to the accompanying drawings. These drawings should not be construed as limiting the present disclosure, but are intended to be exemplary only.

FIG. 1 is a process flowchart illustrating the Correlated Multiphasic Analysis of autosomal DNA matches per USPTO application # Ser. No. 18/793,774.

FIG. 2 illustrates a collection of CMA Test Subjects identified by letter-name designations.

FIG. 3 presents the Test Subject roster and ACS structure utilized by the CMA process.

FIG. 4 presents the Table of Complexes (T° C.) about the nexus of FIG. 2 and the Most Recent Common Ancestral Couple each CMA Test Subject shares with the nexus.

FIG. 5 presents the Top 20 Elements from the ACS of the CMA of the Test Subjects of FIG. 2, sorted by |ACS Class|.

FIG. 6 is a process flowchart illustrating the Reverse-CMA process and the origin of R-CMA's inputs within the CMA process.

FIG. 7 presents the Test Subject roster and ACS structure utilized by the R-CMA process.

FIG. 8 illustrates (left) the Reverse-CMA Table of Complexes (R-CMA T° C.) about our R-CMA nexus and (right) the identical Most Recent Common Ancestral Couple each matching Test Subject shares with the R-CMA nexus.

FIG. 9 illustrates the global structure of the CMA Master Workbook.

FIG. 10 illustrates the data entry fields in the Correlation Worksheet of the Reverse-CMA Workbook, numbered according to the order in which the fields are populated by the end-user.

FIG. 11 presents the summary area of the R-CMA Workbook.

FIG. 12 presents the Tabulation Matrix of the R-CMA Workbook.

FIG. 13 presents a fully curated R-CMA roster from the R-CMA Workbook of FIG. 10.

FIG. 14 illustrates constructed pedigrees of the R-CMA roster's individuals.

### DETAILED DESCRIPTION OF THE INVENTION

### 1. The Reverse-Correlated Multiphasic Analysis Process

### Origins in Correlated Multiphasic Analysis:

Reverse-Correlated Multiphasic Analysis (R-CMA) represents an outgrowth of the concepts and practices employed by Correlated Multiphasic Analysis, and as such it may be helpful at the outset to review the CMA process and how the elements of CMA's Analytic Control Set (ACS) generate the data inputs for Reverse-CMA.

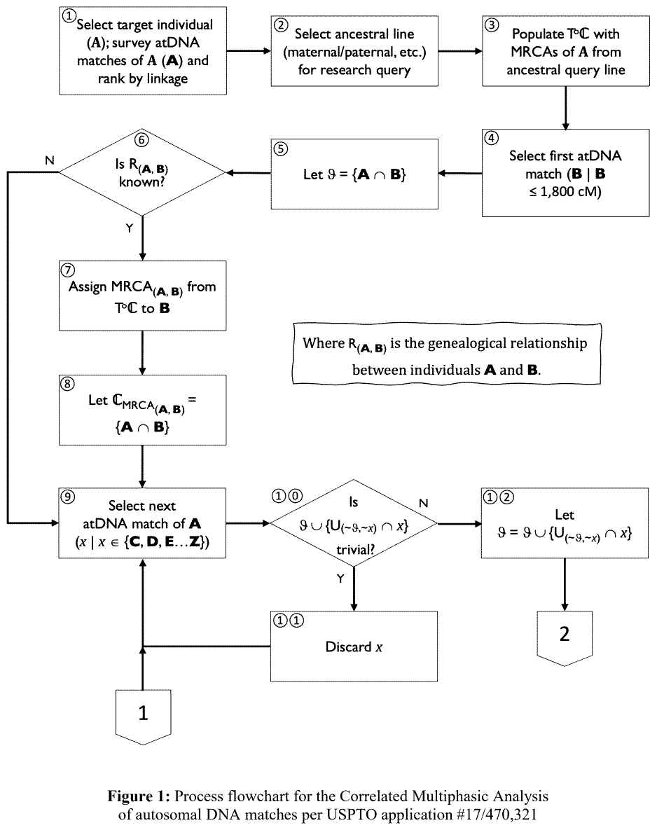

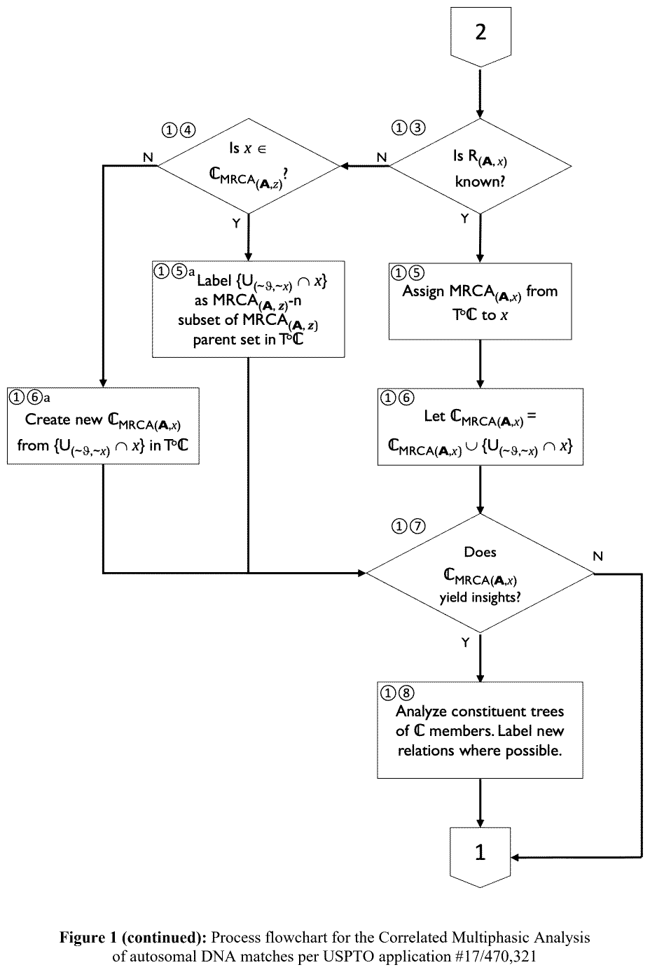

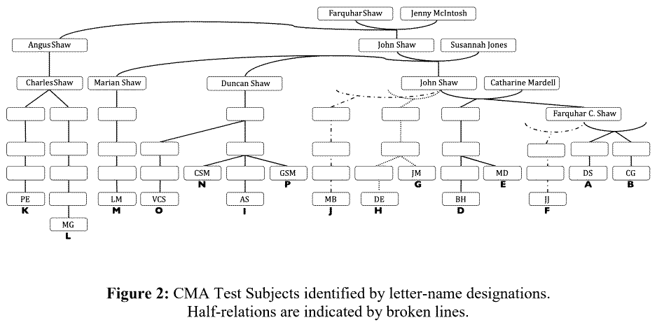

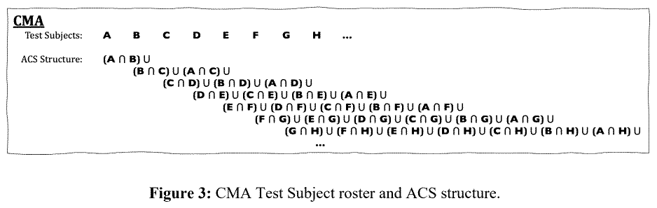

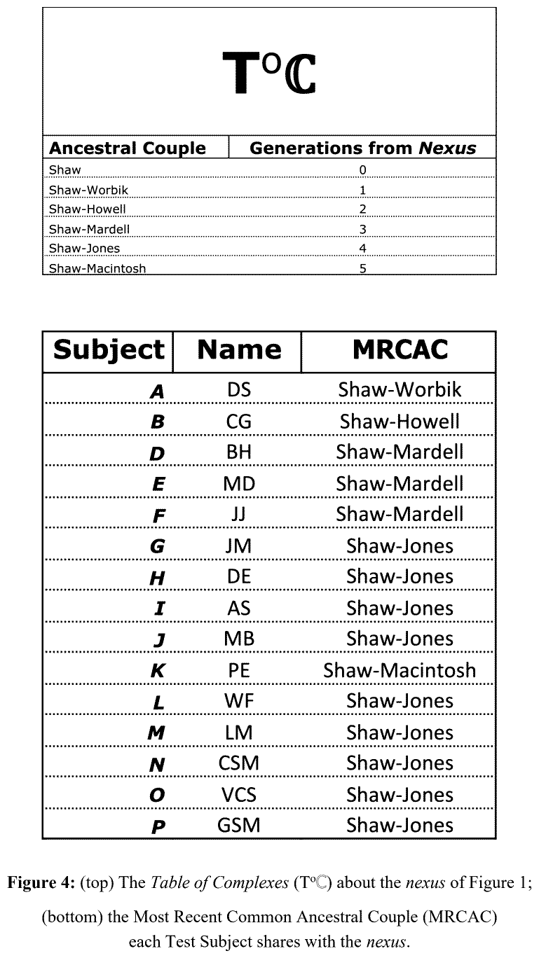

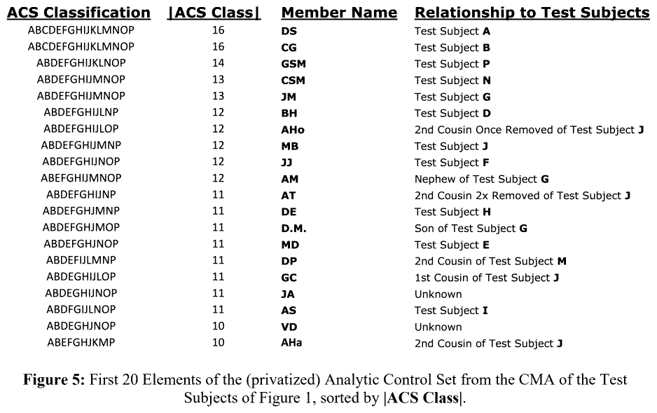

In brief, CMA (FIG. 1) applies set-theoretic operations—primarily union (∪), intersection (∩), and complementation (~)—to a core set of In Common With (ICW) atDNA matches known in the parlance of the CMA process as the Analytic Control Set (ACS).

The ACS may be partitioned into genetic complexes (C): collections of individuals genealogically related to our Test Subjects through the pedigree of a selected “Target Ancestor.” CMA-derived genetic complexes may be fruitfully analyzed using the AASK process after “proofreading” via Shared Match Differentiation (USPTO application # Ser. No. 18/793,774).

Alternatively, the Analytic Control Set—itself a collection of In Common With DNA matches—may be sorted according to the attributes of its constituent elements, including metrics such as the maximum/minimum/average linkage shared with the Test Subjects, or by the number of Test Subjects which share DNA with an individual element of the ACS-what is described as the magnitude of that individual's ACS Classification (|ACS Class|).

Correlated Multiphasic Analysis (CMA) designates an individual as its nexus (FIG. 2, Test Subject A) and employs the DNA matches from a collection of known genealogical relations of the nexus (FIG. 2, Test Subjects B through P) to generate a ranked Analytic Control Set (ACS) from the In Common With matches of its Test Subjects.

FIG. 3 illustrates how CMA's Analytic Control Set is constructed from the Universal Set of the In Common With DNA matches of CMA's Test Subjects. The desktop prototype of the CMA Master Workbook supports a maximum of 25 Test Subjects (B-Z) in addition to the CMA nexus (A).

A Table of Complexes (T° C., FIG. 4, left side) lists the nexus' direct ancestral couples along with the number of generations separating each couple from the nexus individual. Test Subjects B-P are each assigned the Most Recent Common Ancestral Couple (MRCAC, FIG. 4, right side) shared with the nexus. CMA uses this information to assign a genetic complex (C) to each element of the ACS.

The Analytic Control Set is broadly defined once Test Subject data has been entered into the CMA Master Workbook, or processed by an enterprise instantiation of CMA. FIG. 5 presents the first 20 elements of the ACS from the CMA of the Test Subjects of FIG. 2, sorted by the number of Test Subjects each ACS element matches. It's not surprising that many of these ranked ACS elements are the Test Subjects themselves, as indicated in the rightmost column of FIG. 5.

However, the 17^{th} (JA) and 19^{th} (VD) elements of FIG. 5 have no known genealogical connection to our Test Subjects, and therefore represent opportunities for further genealogical research and bioinformatic investigation as the nexus of an R-CMA process.

The CMA process assigns a genetic complex to every element of the ACS. Co-incidentally, both JA and VD are designated as elements of Complex Shaw-Jones (C_{[Shaw-Jones]}), a genetic complex organized about the ancestral couple John Shaw (1774-1819) and his wife Susannah Jones (1781-1859), as shown in FIG. 2. Individuals assigned to this genetic complex are either: descended from one (or both) of John and Susannah, or share an ancestor with either John or Susannah.

While C_{[Shaw-Jones]} provides us with some rough idea as to the genealogical connection of JA and VD to our Test Subjects, there remains much to be ascertained before either JA or VD can be fruitfully connected to the Test Subject hierarchy of FIG. 2. It was for this reason that Reverse CMA (R-CMA) was developed.

### The Reverse-Correlated Multiphasic Analysis Process:

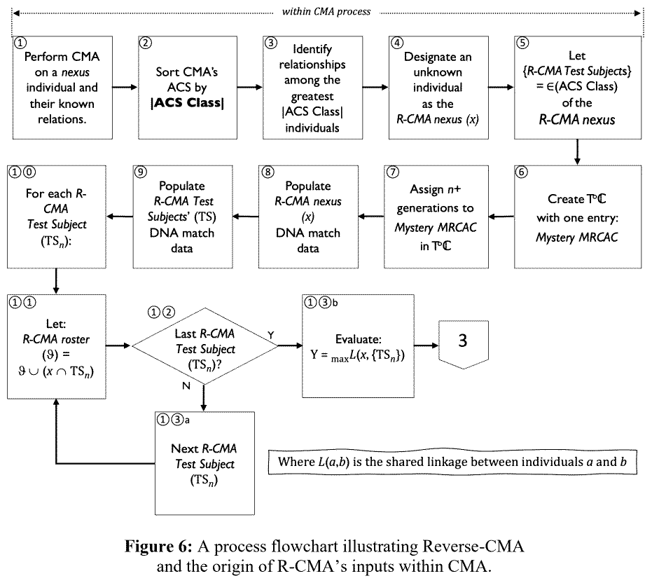

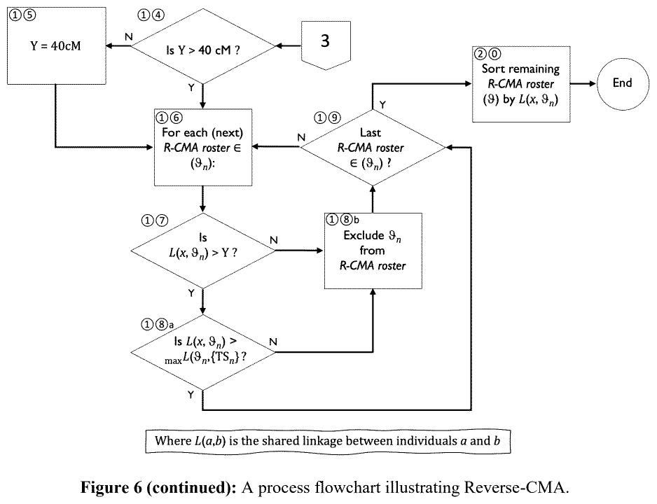

Reverse-Correlated Multiphasic Analysis (FIG. 6) inverts the CMA process, employing Axiomatic Set Theory to generate a collection of In Common With (ICW) DNA matches that reflect the ancestral family lines connecting the R-CMA nexus to our Test Subjects. R-CMA further curates these ICW matches, to remove individuals directly descended from the Test Subjects' MRCAC, as well as small centimorgan matches whose origins remain ambiguous.

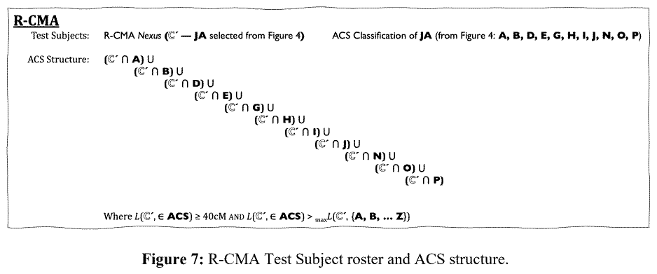

In addition to the full set of DNA matches of its R-CMA nexus individual, Reverse-CMA takes as its inputs the DNA matches of each CMA Test Subject listed in the ACS Classification of the R-CMA nexus. As the R-CMA nexus of FIG. 7 matches Test Subjects A, B, D, E, G, H, I, J, N, O, and P, the R-CMA process will require eleven full sets of DNA matches (those of the aforementioned Test Subjects) in addition to the (newly-acquired) DNA matches of the R-CMA nexus itself.

While the likelihood that any of the Test Subjects matching the R-CMA nexus forms an unfavorable shared match trio with the R-CMA nexus and another Test Subject is slim to none, the favorability of each matching test subject may be ascertained using the Shared Match Differentiation process (USPTO application # Ser. No. 18/793,774).

Despite similar data inputs, CMA and Reverse-CMA process their inputs to form an Analytic Control Set (ACS) in a distinct manner. FIG. 7 presents the ACS structure of the Reverse-CMA of individual JA from FIG. 5. Unlike CMA, which assembles a Universal Set of In Common With matches as its ACS (FIG. 3), every element of R-CMA's ACS (FIG. 7) must include the R-CMA nexus.

R-CMA further refines its Analytic Control Set to exclude individuals descended from its Test Subjects' MRCAC as well as to exclude ICW matches with linkage of less than 40 cM whose ancestral origins are likely to remain ambiguous until the R-CMA process has run its course.

This R-CMA Matching Threshold (Y, in FIG. 6, steps \textcircled{1}\textcircled{3} to \textcircled{1}\textcircled{7} is defined as the maximum linkage shared between the R-CMA nexus and any of its Test Subjects, or 40 cM, whichever is greater. All elements of R-CMA's Analytic Control Set which fall below this threshold are excluded from the end product of the R-CMA process.

The end product of Reverse-CMA is the R-CMA roster: a collection of individuals who share a common ancestral line, descended from a “Mystery Most Recent Common Ancestral Couple” distinct from—but more closely related to—the MRCAC of our R-CMA Test Subjects.

Beginning with the verified pedigrees of individuals most closely related to the R-CMA nexus, researchers may employ traditional practices to identify the ancestral line shared by the less closely related individuals of the R-CMA roster, and thereby connect the R-CMA nexus to more distantly related members of the roster—the idea being that the MRCAC shared by the R-CMA roster's might be more readily connected to the pedigree of the CMA Testing Subjects through shared surnames, localities and dates.

### II. Reverse-Cma on the Desktop Computing Platform Via the Reverse-Cma Workbook:

Reverse-CMA designates an individual of unknown (or uncertain) connection to our Test Subjects as its nexus. From FIG. 5, we'll select individual JA as the R-CMA nexus through which we'll demonstrate R-CMA using the Reverse-CMA Workbook, a scripted environment which runs in Microsoft Excel.

In our initial CMA process, individual JA was assigned to C_{[Shaw-Jones]}—4 generations removed from our CMA nexus. In our Reverse-CMA, JA is our R-CMA nexus, and we know only that JA shares an unknown ancestral couple—a “Mystery MRCAC”—with our test subjects. JA's closest relation to our CMA Test Subjects would be if JA were descended from either John Shaw or Susannah Jones—in which case Shaw-Jones would be our MRCAC, and each test subject would be also be at minimum 4 generations removed form JA.

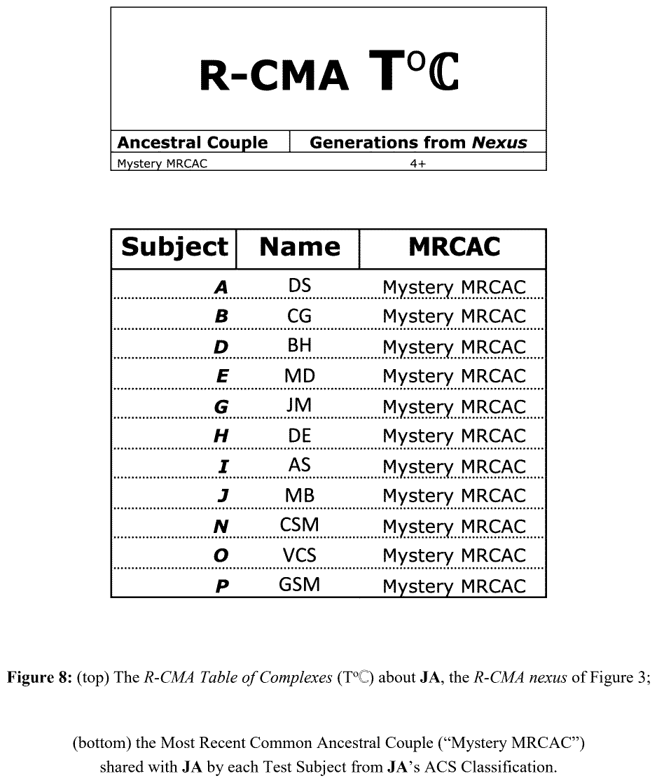

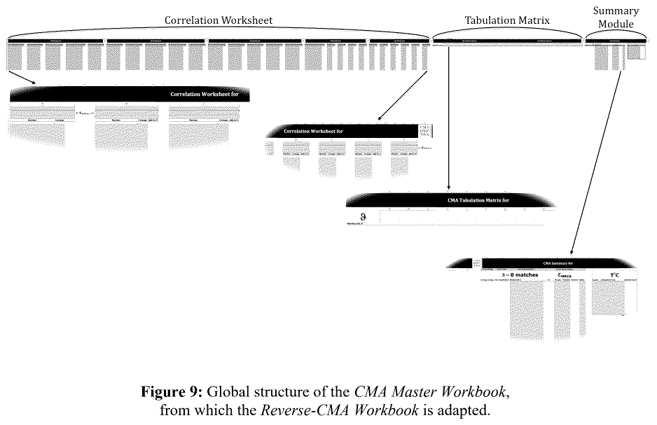

However, if JA shares a common ancestor with either John or Susannah, then our Mystery MRCAC would be farther removed than 4 generations, and so our R-CMA Table of Complexes indicates that our mystery MRCAC is “4+” generations removed from our nexus (FIG. 8, left). As a point of fact, because our Mystery MRCAC is the only entry in our R-CMA Table of Complexes, we could assign any positive integer to our “Generations from Nexus” value, but we have chosen “4+” to remain consistent with our initial CMA process.

Whereas CMA utilized a full complement of Test Subjects, each sharing their own MRCAC with our nexus individual (as shown in FIG. 3), Reverse-CMA is organized about our R-CMA nexus (in our example, JA) and uses only the Test Subjects JA matched in our initial CMA process (A, B, D, E, G, H, I, J, N, O, and P—FIG. 5, leftmost column). Therefore, each R-CMA Test subject shares the same “Mystery MRCAC” with JA (FIG. 8, right).

Whereas our CMA process employed the CMA Master Workbook (FIG. 9) to tabulate the full sets of DNA matches of our nexus and Test Subjects, Reverse-CMA modifies the CMA Master Workbook prototype to create the Reverse-CMA Workbook (FIGS. 8, 9, 10) to perform its analysis. The Reverse-CMA Workbook requires full sets of DNA matches of our R-CMA nexus and the DNA matches of those Test Subjects which share DNA with our R-CMA nexus—the elements of the R-CMA nexus' ACS Classification (Subjects A, B, D, E, G, H, I, J, N, O, and P).

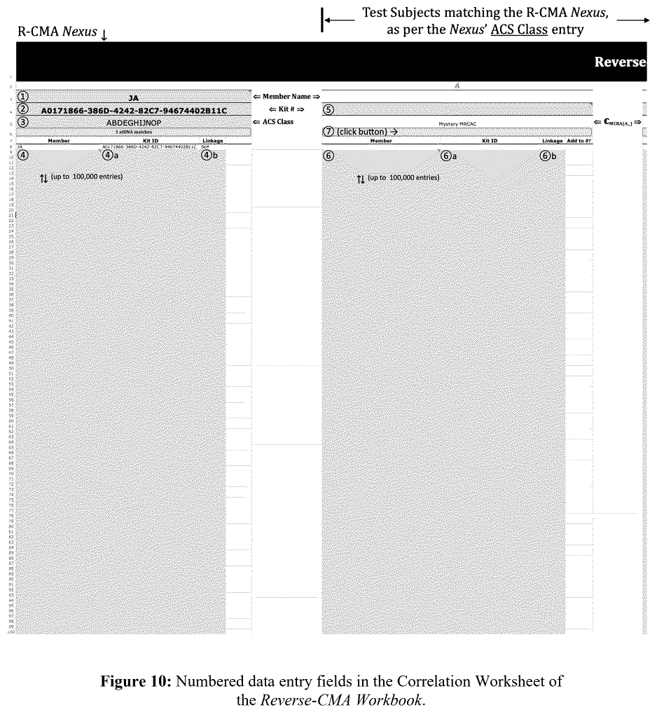

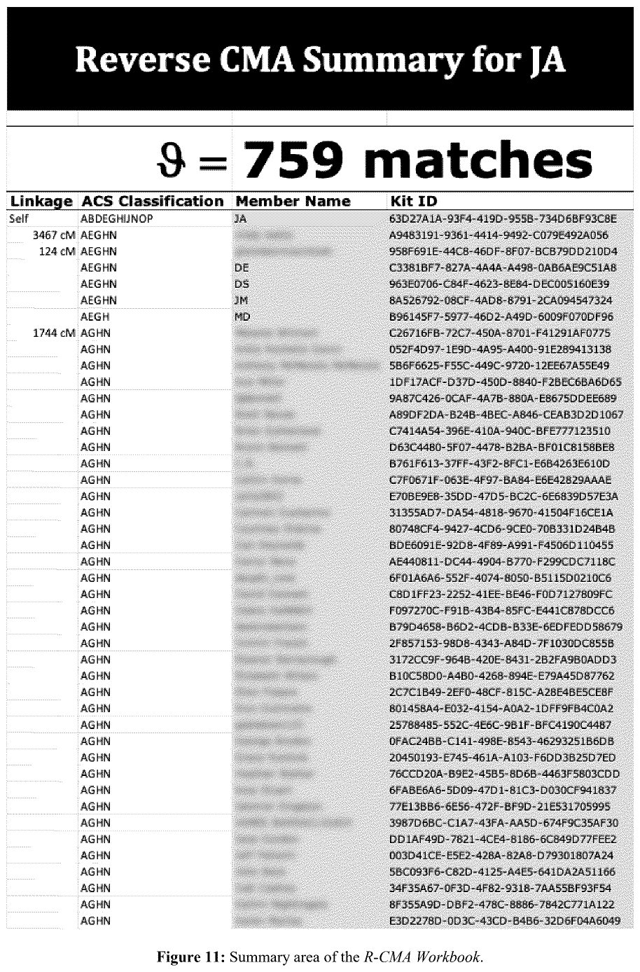

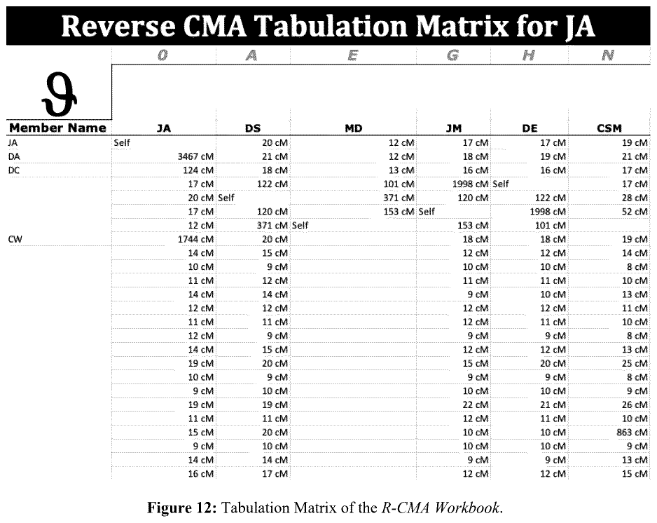

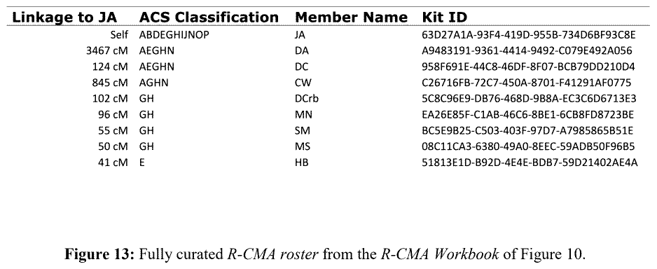

The data entry fields of the Correlation Worksheet of the Reverse-CMA Workbook of FIG. 10 are numbered according to the order in which they are populated by the end-user:
\textcircled{1}: Enter the Name of the R-CMA nexus.
\textcircled{2}: Enter the Test Kit ID of the R-CMA nexus.
\textcircled{3}: Enter the ACS Classification associated with the R-CMA nexus from the CMA process.
\textcircled{4}: Using the downloaded DNA matches of the R-CMA nexus, paste the values of the:
   Names of the R-CMA nexus' DNA matches
   a) Test Kit IDs of the R-CMA nexus' DNA matches
   b) Linkage shared by the R-CMA nexus' DNA matches

Recalculate the Reverse-CMA Workbook. Test Subject letter-names (A, B, etc.) will populate above each Test Subject's data entry column.
\textcircled{5}: Enter the Test Kit ID of the Test Subject corresponding to the letter name above the unpopulated data column to the right of the R-CMA nexus' data.
\textcircled{6}: Using the downloaded DNA matches of the 1^{st} matching Test Subject, paste the values of the:
   Names of the Test Subject's DNA matches
   a) Test Kit IDs of the Test Subject's DNA matches
   b) Linkage shared by the Test Subject's DNA matches

### Recalculate the Reverse-CMA Workbook.

\textcircled{7}: Click the blue button labelled with the number of DNA matches belonging to the Test Subject: “##, ### at DNA matches . . . ” A programmed script will read through the Test Subject's DNA matches and recalculate the Reverse-CMA Workbook once more.
\textcircled{8}-\textcircled{1}\textcircled{0}: Repeat steps \textcircled{5}, \textcircled{6}, and \textcircled{7} for each Test Subject corresponding to the letter-name labels at the top of each data entry column.

After steps \textcircled{5}, \textcircled{6}, and \textcircled{7} have been performed for the DNA matches of each element of the R-CMA nexus' ACS Classification, the R-CMA roster may be previewed by scrolling all the way to the rightmost column of the Reverse-CMA Workbook to view the Reverse-CMA Summary (FIG. 11, individual names have been obscured for privacy reasons).

The Reverse-CMA Summary of FIG. 11 presents the R-CMA roster in an unabridged format, listing every In Common With match in which the R-CMA nexus is a participant. The R-CMA process curates this list, however, excluding individuals descended from the MRCAC of the CMA Test Subjects, and also individuals whose shared linkage is so small (less than 40 cM) so as to be ambiguous as to its ancestral origins.

Procedurally, the Reverse-CMA Workbook curates the R-CMA roster by excluding In Common With matches which share more DNA with the CMA Test Subjects than with the R-CMA nexus. The Reverse-CMA Workbook utilizes the Tabulation Matrix portion of its main worksheet (FIG. 12) which cross-references the In Common With matches of the R-CMA roster against the amount of DNA shared with each CMA Test Subject.

In Common With matches whose linkage with the R-CMA nexus falls below lower reporting limit are also excluded from the R-CMA roster. This limit is defined as 40 cM, or the maximum linkage the R-CMA nexus shares with any of the CMA Test Subjects—whichever is greater.

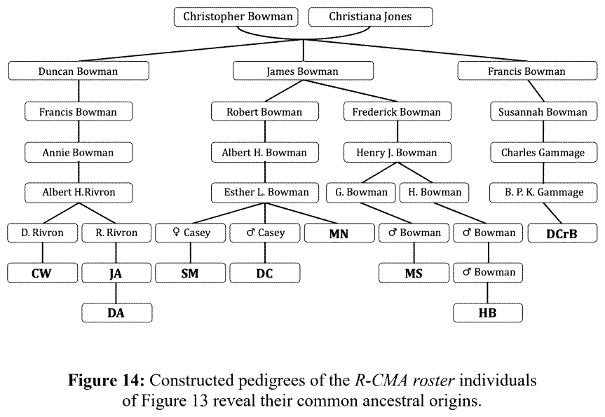

The curated R-CMA roster may be reviewed by clicking on the [Output Summary to PDF] button in FIG. 11. (The curated R-CMA roster for our example is shown in FIG. 13.)

The curated R-CMA roster provides actionable intelligence to the genealogical researcher in that the individuals of the roster share a Most Recent Common Ancestral Couple through which the roster (including the R-CMA nexus) connects to the MRCAC of our CMA Test Subjects. The most useful strategy here is to construct pedigrees for the individuals listed in the R-CMA roster, beginning with those individuals which share greatest linkage with the R-CMA nexus. Some of these relationships will be obvious—for instance, DA can only be JA's parent or child—but other relationships will require investigation using traditional genealogical research methods. As with other collections derived from shared matches, the elements of the R-CMA roster may be validated using the SMD process.

FIG. 14 presents an inheritance diagram showing how the 9 individuals of our R-CMA roster each descend from Christiana Jones (1787-1871), whom along with her husband Christopher Bowman are the Most Recent Common Ancestral Couple of our R-CMA roster. Further research is required to determine precisely how this Bowman-Jones couple are related to the Shaw-Jones MRCAC from which our CMA Test Subjects are descended, but one obvious possibility is that Christiana is the younger sister of Susannah Jones (1781-1859) of FIG. 2.

1. A process for performing Reverse-Correlated Multiphasic Analysis (R-CMA) of autosomal DNA (atDNA) matches, independent of any specific DNA testing provider or tabulating mechanism.

2. The process of claim 1, whereby an R-CMA nexus is identified by ranking the elements of the Analytic Control Set (ACS) of an existing CMA by the magnitude of each element's ACS Classification (|ACS Class|) and selecting an otherwise unidentified ACS element with a large |ACS Class|.

3. The process of claim 1, whereby the R-CMA Test Subjects are derived from the elements of the R-CMA Nexus' ACS Classification: {R-CMA Test Subjects}=∈(ACS Class)_{R-CMA Nexus}.

4. The process of claim 1, whereby the nature of the R-CMA Test Subjects' shared matches with the R-CMA nexus may optionally be evaluated using the Shared Match Differentiation process.

5. The process of claim 1, whereby the number of generations separating the R-CMA Nexus from the Most Recent Common Ancestral Couple (MRCAC) shared by the Test Subjects and the R-CMA Nexus is assigned an arbitrarily large value (n+), greater than the number of generations separating the R-CMA Test Subjects from their own MRCAC.

6. The process of claim 1, whereby the R-CMA roster (the Analytic Control Set of the R-CMA process) is constructed from the union of the In Common With matches of the R-CMA nexus' DNA matches and the DNA matches of each of the Test Subjects.

7. The process of claim 1, whereby the R-CMA Matching Threshold is defined as the maximum linkage shared by the R-CMA nexus and any of the Test Subjects, or 40 cM, whichever is greater.

8. The process of claim 1, whereby the elements of the R-CMA roster are filtered to exclude those elements which do not exceed the Matching Threshold.

9. The process of claim 1, whereby the elements of the R-CMA roster are filtered to exclude those elements which share more DNA with the Test Subjects than with the R-CMA nexus.

10. The process of claim 1, whereby the curated R-CMA roster is sorted by the linkage each element of the roster shares with the R-CMA nexus.
